SEQUENCE LISTING (32 sequences)

SEQ ID NO: 1 - RNA, 23 nt, synthetic construct
  misc_feature 1..23: note = Synthetic: bta-miR-19b
  source 1..23: mol_type = other RNA
tgtgcaaatccatgcaaaactga

SEQ ID NO: 2 - RNA, 23 nt, synthetic construct
  misc_feature 1..23: note = Synthetic: bta-miR-196b
  source 1..23: mol_type = other RNA
taggtagtttcctgttgttggga

SEQ ID NO: 3 - RNA, 24 nt, synthetic construct
  misc_feature 1..24: note = Synthetic: bta-miR-146a
  source 1..24: mol_type = other RNA
tgagaactgaattccataggttgt

SEQ ID NO: 4 - RNA, 23 nt, synthetic construct
  misc_feature 1..23: note = Synthetic: bta-miR-29b
  source 1..23: mol_type = other RNA
tagcaccatttgaaatcagtgtt

SEQ ID NO: 5 - RNA, 23 nt, synthetic construct
  misc_feature 1..23: note = Synthetic: bta-miR-29a
  source 1..23: mol_type = other RNA
ctagcaccatctgaaatcggtta

SEQ ID NO: 6 - RNA, 22 nt, synthetic construct
  misc_feature 1..22: note = Synthetic: bta-miR-142-3p
  source 1..22: mol_type = other RNA
agtgtttcctactttatggatg

SEQ ID NO: 7 - RNA, 23 nt, synthetic construct
  misc_feature 1..23: note = Synthetic: bta-miR-155
  source 1..23: mol_type = other RNA
ttaatgctaatcgtgataggggt

SEQ ID NO: 8 - RNA, 23 nt, synthetic construct
  misc_feature 1..23: note = Synthetic: bta-miR-582
  source 1..23: mol_type = other RNA
ttacagttgttcaaccagttact

SEQ ID NO: 9 - RNA, 24 nt, synthetic construct
  misc_feature 1..24: note = Synthetic: bta-miR-21-5p
  source 1..24: mol_type = other RNA
tagcttatcagactgatgttgact

SEQ ID NO: 10 - RNA, 23 nt, synthetic construct
  misc_feature 1..23: note = Synthetic: bta-miR-6517
  source 1..23: mol_type = other RNA
tcagggtccgtgagctcctcggc

SEQ ID NO: 11 - RNA, 22 nt, synthetic construct
  misc_feature 1..22: note = Synthetic: bta-miR-7857-5p
  source 1..22: mol_type = other RNA
atagccagttggggaagaatgc

SEQ ID NO: 12 - RNA, 23 nt, synthetic construct
  misc_feature 1..23: note = Synthetic: bta-miR-24
  source 1..23: mol_type = other RNA
gtgcctactgagctgatatcagt

SEQ ID NO: 13 - RNA, 21 nt, synthetic construct
  misc_feature 1..21: note = Synthetic: bta-miR-378c
  source 1..21: mol_type = other RNA
actggacttggagtcagaagt

SEQ ID NO: 14 - RNA, 22 nt, synthetic construct
  misc_feature 1..22: note = Synthetic: bta-miR-1271
  source 1..22: mol_type = other RNA
cttggcacctagtaagtactca

SEQ ID NO: 15 - RNA, 22 nt, synthetic construct
  misc_feature 1..22: note = Synthetic: bta-miR-100
  source 1..22: mol_type = other RNA
aacccgtagatccgaacttgtg

SEQ ID NO: 16 - RNA, 25 nt, synthetic construct
  misc_feature 1..25: note = Synthetic: bta-miR-301a
  source 1..25: mol_type = other RNA
cagtgcaatagtattgtcaaagcat

SEQ ID NO: 17 - RNA, 23 nt, synthetic construct
  misc_feature 1..23: note = Synthetic: bta-miR-32
  source 1..23: mol_type = other RNA
tattgcacatgactaagttgcat

SEQ ID NO: 18 - RNA, 22 nt, synthetic construct
  misc_feature 1..22: note = Synthetic: bta-miR-1247-5p
  source 1..22: mol_type = other RNA
acccgtcccgtgcgtccccgga

SEQ ID NO: 19 - RNA, 22 nt, synthetic construct
  misc_feature 1..22: note = Synthetic: bta-miR-184
  source 1..22: mol_type = other RNA
tggacggagaactgataagggt

SEQ ID NO: 20 - RNA, 21 nt, synthetic construct
  misc_feature 1..21: note = Synthetic: bta-miR-202
  source 1..21: mol_type = other RNA
ttcctatgcatatacttcttt

SEQ ID NO: 21 - RNA, 23 nt, synthetic construct
  misc_feature 1..23: note = Synthetic: bta-miR-137
  source 1..23: mol_type = other RNA
ttattgcttaagaatacgcgtag

SEQ ID NO: 22 - RNA, 23 nt, synthetic construct
  misc_feature 1..23: note = Synthetic: bta-miR-105a
  source 1..23: mol_type = other RNA
tcaaatgctcagactcctgtggt

SEQ ID NO: 23 - RNA, 22 nt, synthetic construct
  misc_feature 1..22: note = Synthetic: bta-miR-433
  source 1..22: mol_type = other RNA
atcatgatgggctcctcggtgt

SEQ ID NO: 24 - RNA, 22 nt, synthetic construct
  misc_feature 1..22: note = Synthetic: bta-miR-133b
  source 1..22: mol_type = other RNA
tttggtccccttcaaccagcta

SEQ ID NO: 25 - RNA, 23 nt, synthetic construct
  misc_feature 1..23: note = Synthetic: bta-miR-20a-5p
  source 1..23: mol_type = other RNA
taaagtgcttatagtgcaggtag

SEQ ID NO: 26 - RNA, 22 nt, synthetic construct
  misc_feature 1..22: note = Synthetic: bta-miR-93
  source 1..22: mol_type = other RNA
caaagtgctgttcgtgcaggta

SEQ ID NO: 27 - RNA, 22 nt, synthetic construct
  misc_feature 1..22: note = Synthetic: bta-miR-16a-5p
  source 1..22: mol_type = other RNA
tagcagcacgtaaatattggtg

SEQ ID NO: 28 - RNA, 24 nt, synthetic construct
  misc_feature 1..24: note = Synthetic: bta-miR-17-5p
  source 1..24: mol_type = other RNA
caaagtgcttacagtgcaggtagt

SEQ ID NO: 29 - RNA, 22 nt, synthetic construct
  misc_feature 1..22: note = Synthetic: bta-miR-92a
  source 1..22: mol_type = other RNA
tattgcacttgtcccggcctgt

SEQ ID NO: 30 - RNA, 22 nt, synthetic construct
  misc_feature 1..22: note = Synthetic: oan-miR-7417-5p
  source 1..22: mol_type = other RNA
ttccccactctgagcacacagc

SEQ ID NO: 31 - RNA, 23 nt, synthetic construct
  misc_feature 1..23: note = Synthetic: cel-miR-70-3p
  source 1..23: mol_type = other RNA
taatacgtcgttggtgtttccat

SEQ ID NO: 32 - RNA, 22 nt, synthetic construct
  misc_feature 1..22: note = Synthetic: ath-miR-167d
  source 1..22: mol_type = other RNA
tgaagctgccagcatgatctgg

(19) United States
(12) Patent Application Publication
(10) Pub. No.: US 2024/0352525 A1
(43) Pub. Date: Oct. 24, 2024

(54) MICRORNA ASSAY FOR DETECTION OF JOHNE'S INFECTION AND DISEASE

(71) Applicant: MI:RNA Ltd., Edinburgh (GB)

(72) Inventors: Eve HANKS, Edinburgh (GB); Robert COULTOUS, Whitburn (GB)

(73) Assignee: MI:RNA Ltd., Edinburgh (GB)

(21) Appl. No.: 18/639,850

(22) Filed: Apr. 18, 2024

Related U.S. Application Data
(60) Provisional application No. 63/496,822, filed on Apr. 18, 2023.

(51) Int. Cl. C12Q 1/6883

(52) U.S. Cl. CPC C12Q 1/6883; C12Q 2600/158; C12Q 2600/178

(57) ABSTRACT

A method is provided for detecting the presence of mycobacterial infection or disease in a subject, comprising the steps of determining the level of expression of each of a plurality of miRNAs within a sample from a subject and using one or more Artificial Intelligence (AI) model to predict the disease condition of the subject.

### REFERENCE TO RELATED APPLICATIONS

This application claims benefit to and priority to U.S. Provisional Application No. 63/496,822, filed on Apr. 18, 2023, which is hereby incorporated by this reference in its entirety.

### REFERENCE TO ELECTRONIC SEQUENCE LISTING

The application contains a Sequence Listing which has been submitted electronically in .XML format and is hereby incorporated by reference in its entirety. Said .XML copy, created on May 9, 2024, is named “068075.001US.xml” and is 41,720 bytes in size. The sequence listing contained in this .XML file is part of the specification and is hereby incorporated by reference herein in its entirety.

### FIELD

The present invention relates to isolated nucleic acid molecules known as microRNAs (miRNAs) and miRNA precursor molecules and their use in diagnosis and therapy. The invention also relates to a method and a kit for diagnosing a Mycobacterial disease such as Johne's disease (Mycobacterial avium Subsp. Paratuberculosis).

### BACKGROUND

Biomarkers have the potential to allow for early diagnosis, risk stratification and therapeutic management of various diseases. Although research into the use of biomarkers has developed in recent years, the clinical translation of disease biomarkers as endpoints in disease management and in the development of diagnostic products still poses a challenge.

miRNAs are a class of small non-coding RNAs which have been identified as having the potential to act as biomarkers. miRNAs were first discovered in the free-living nematode Caenorhabditis elegans where it was found that small, non-coding RNAs known as lin-4 and let-7 were responsible for regulating the expression of developmental proteins in C. elegans through suppression of messenger RNA (mRNA) levels (Wightman, et al., 1993; Lee, et al., 1993; Lee & Ambros, 2001). miRNAs bind predominantly to the three prime (3′) untranslated region (UTR) of their target genes resulting in suppression of translation and/or mRNA degradation. Coutinho et al (2007) analyzed bovine immunity and embryonic tissues and reported that miRNAs are frequently conserved across species. In addition, it was found that some miRNAs are expressed preferentially in specific tissue types while others are expressed more uniformly across different tissues.

miRNAs have been identified as key regulators of the immune system of many organisms (Mehta & Baltimore, 2016). They are recognized as key mediators of innate immunity (Momen-Heravi & Bala, 2018), the first line of defense, and adaptive immunity (Jia, et al., 2014) which is a specific response to a pathogen. This makes the use of miRNAs particularly interesting since understanding their expression will allow for a greater understanding of the epigenetic responses to disease, wherein the diseases are both infectious and non-infectious in origin (Rupaimoole & Slack, 2017). It was subsequently discovered that miRNAs are released from tissues into the systemic circulation and can be found in other biofluids (for example, in a blood sample). The term ‘liquid biopsy’ was thus adopted (Giannopoulou, et al., 2019). Furthermore, miRNAs also offer a potential as therapeutic targets. If miRNAs are dysregulated in disease states then it is considered that controlling their expression and encouraging healing over inflammation would be beneficial for patients. This idea has been termed anti-miRNAs (Piotto, et al., 2018).

Mycobacterium is a genus of over 190 species in the phylum Actinomycetota, assigned its own family, Mycobacteriaceae. The genus Mycobacterium includes pathogens known to cause serious diseases in mammals, including, for example, tuberculosis and leprosy. Mycobacterium avium subspecies paratuberculosis is a bacterial parasite and the causative agent of paratuberculosis/Johne's disease, a disease predominately found in cattle and sheep. Infection with this microorganism results in substantial farming economic losses and animal morbidity.

Johne's disease is an incurable, infectious, wasting disease of cattle, sheep and other livestock species which is fatal. The causative agent, Mycobacterium avium subspecies paratuberculosis (MAP), is endemic in 40-60% of beef farms and 70% of dairy farms (Woodbine et al, 2009) in the UK causing significant economic losses, particularly in dairy and beef farming. Animals are typically infected early in life and infection leads to weight loss, reduced milk yield in dairy animals of around 20% (Benedictus et al, 1987), high somatic cell counts of >100,000, susceptibility to other disease and problems with reproduction (Tiwari et al, 2003, and reduction of carcass weight by up to 40% (Ott et al, 1999). Increased greenhouse gas emissions, of up to 40% for each unit of meat, have been suggested for infected cows (DEFRA, 2015).

Clinical disease takes 2-5 years to develop. The currently available ELISA test has a sensitivity as low as 15% in early stages of the disease prior to clinical signs (Matthews et al, 2021). Fecal culture is more sensitive but can take up to 8-12 weeks due to the slow growing nature of the MAP bacteria (Matthews et al, 2021). Current testing cannot reliably detect subclinical disease and cannot predict disease course to help identify which animals will progress to sub clinical and clinical states. Often a combination of testing modalities is required, increasing costs and allowing the infected animals to spread disease throughout the farm. Therefore, Johne's disease is impossible for farmers to control due to an inability to diagnose early-stage disease as tests lack sensitivity, rendering a ‘test and cull’ approach ineffective.

Additionally, there are concerns surrounding the zoonotic potential of MAP in human health with the causative agent linked to Crohn's disease, Multiple Sclerosis and Diabetes type 2 (McNees et al, 2015). The bacteria can be found viable in pasteurized milk

The loss of stock due to this disease is estimated to cost in the region of £10 million per year to the UK agricultural economy but the true costs of the disease will be much higher (Bennett & IJpelaar, 2005).

Not all infected livestock will progress to clinical disease, so diagnosis of pathogen infection is not a predictor of clinical disease. It is established that resilience to clinical progress is down to the individual host response, but the mechanisms for this are not understood

miRNA profiles are thought to hold substantial amounts of information and are conserved across species such as farm animals, horses, companion animals and humans. So far, miRNAs have been mainly studied in tissue material where it has been found that miRNAs are expressed in a highly tissue-specific manner. In order to improve the biomarker capabilities in diagnosis there is a need for disease specific, well performing biomarkers such as miRNA biomarkers.

Therefore, it is the aim of the present invention to provide a method for diagnosing a Mycobacterial disease such as Johne's disease using miRNA biomarkers Provide herein are methods of diagnosis by detecting and analyzing variations in microRNAs (miRNAs) in fluids such as urine, milk, serum or plasma. More particularly, the invention provides a method for in vitro diagnosis of Mycobacterium; evaluation of disease stage; monitoring of the progression of disease; evaluation of disease complications and relapse; prognosis; and evaluation of drug efficacy and therapeutic effects.

### SUMMARY

In accordance with the purpose(s) of this invention, as embodied and broadly described herein, this invention, according to a first aspect, provides a method for detecting the presence of Johne's disease in a subject, comprising the steps of: (a) determining the level of expression of each of a plurality of miRNAs within a sample from a subject; and (b) comparing the level of expression of each miRNA molecule with at least one pre-determined reference level characteristic of a non-diseased subject for each one of the plurality of the miRNA molecules of step (a), wherein a deviation of the level of expression of said miRNA molecules from step (a) in comparison with the at least one reference level allows for the diagnosis and/or prognosis of the disease.

Preferably, the plurality of miRNA molecules comprise of bta-miR-19b, bta-miR-196b, bta-miR-146a, bta-miR-29b, bta-miR-29a, bta-miR-142-3p, bta-miR-155, bta-miR-582, bta-miR-21-5p, bta-miR-6517, bta-miR-7857-5p, bta-miR-24, bta-miR-378c, bta-miR-1271, bta-miR-100, bta-miR-301a, bta-miR-32, bta-miR-1247-5p, bta-miR-184, bta-miR-202, bta-miR-137, bta-miR-105a, bta-miR-433, bta-miR-133b, bta-miR-93, bta-miR-20a-5p, bta-miR-16a-5p, bta-miR-17-5p, bta-miR-92a, oan-miR-7417-5p, cel-miR-70-3p and ath-miR-167d.

Preferably, the subject is an animal. Typically, the subject is a mammal.

It is an advantage of the invention that the method provides an accurate and useful test that can be used in veterinary practice. It is known that certain levels of expression of certain miRNA molecules can indicate the presence of disease. However, measuring the level of expression of the plurality of miRNA molecules in accordance with the invention allows for the accurate diagnosis of disease within a subject. The determination of disease within the context of the present invention would not be possible with one biomarker because it is not simply the increase or decrease of one marker that provides the diagnostic information. Rather, it is the differential expression of the plurality of miRNAs in relation to each other and the pattern recognition of the plurality of miRNAs that enables the disease detection.

Typically, the method further comprises the use of at least one normalizer and/or control miRNA molecule. Preferably, the control miRNA molecule is an off-species control miRNA molecule.

Preferably, at least one normalizer is selected from the group consisting of bta-miR-93, bta-miR-16a-5p, bta-miR-17-5p and bta-miR-92a. Preferably, the at least one off-species control is selected from the group consisting of oan-miR-7417-5p, cel-miR-70-3p and ath-miR-167d

Preferably, at least one normalizer is used to ‘normalize’ data, i.e. to control for variation between the samples tested in the method of the invention, and the at least one control is used to try to ensure there are no failure or false readings in the results. Preferably, at least one off-species control is added in to show that the miRNAs detected are relevant to the target species of the panel. Preferably, the off-species control is an miRNA from another species, i.e. non ruminant. Advantageously, the use of at least one off-species control provides another layer of control to distinguish between background or non-specific signals and a positive result (for example, indicating the presence of disease in a subject).

Typically, the disease is selected from the group caused species within the Mycobacterium genus and related conditions.

In one embodiment, the reference level may be provided by comparing the level of miRNA expression from the sample with an miRNA expression level from an unaffected control and a sample from a diseased animal.

Preferably, the sample is a biofluid selected from the group consisting of blood, urine, milk, tissue fluid, saliva, cerebrospinal fluid (CSF), feces or another biofluid.

Preferably, the miRNAs are cell free miRNAs.

Advantageously, the method allows for high throughput, low cost testing that can be carried out and completed in a reasonable timeframe.

It is an advantage of the invention that the method that this diagnostic test is the first test of its kind with a unique ability to diagnose Johne's disease in cattle in the early stages of the infection and then predict the likelihood of progression to clinical stages with higher sensitivity (up to 95% diagnostic accuracy is predicted from preliminary data analysis). A unique miRNA panel of up to 32 miRNAs are used to screen small amounts of biofluid for upregulation or downregulation in these markers (FIG. 1). Trial data in 131 cows (65 healthy, 66 Johne's infected) has demonstrated a sensitivity of 69%, a specificity of 75% and an accuracy of 72% (Table 4).

According to another aspect, there is provided a kit for use in performing the method of the first aspect comprising means for determining the level of expression of each one of the following miRNA molecules: bta-miR-19b, bta-miR-196b, bta-miR-146a, bta-miR-29b, bta-miR-29a, bta-miR-142-3p, bta-miR-155, bta-miR-582, bta-miR-21-5p, bta-miR-6517, bta-miR-7857-5p, bta-miR-24, bta-miR-378c, bta-miR-1271, bta-miR-100, bta-miR-301a, bta-miR-32, bta-miR-1247-5p, bta-miR-184, bta-miR-202, bta-miR-137, bta-miR-105a, bta-miR-433, bta-miR-133b, bta-miR-93, bta-miR-20a-5p, bta-miR-16a-5p, bta-miR-17-5p, bta-miR-92a, oan-miR-7417-5p, cel-miR-70-3p and ath-miR-167d.

According to another aspect, there is provided a method of selecting a panel for use in disease diagnosis comprising the steps of: (a) selecting a group of miRNA molecules the differential expression of which may be associated with a disease condition; (b) predicting the disease condition based on a deviation of the level of expression of said miRNA molecules from step (a) and (b); and (c) reducing the number of miRNAs in the panel to a minimum number to provide a panel of miRNAs that still produces a result.

Preferably, the group of miRNA molecules comprise of bta-miR-19b, bta-miR-196b, bta-miR-146a, bta-miR-29b, bta-miR-29a, bta-miR-142-3p, bta-miR-155, bta-miR-582, bta-miR-21-5p, bta-miR-6517, bta-miR-7857-5p, bta-miR-24, bta-miR-378c, bta-miR-1271, bta-miR-100, bta-miR-301a, bta-miR-32, bta-miR-1247-5p, bta-miR-184, bta-miR-202, bta-miR-137, bta-miR-105a, bta-miR-433, bta-miR-133b, bta-miR-93, bta-miR-20a-5p, bta-miR-16a-5p, bta-miR-17-5p, bta-miR-92a, oan-miR-7417-5p, cel-miR-70-3p and ath-miR-167d.

According to yet another aspect, there is provided a method of selecting a panel for use in differential diagnosis between a symptom-free M. paratuberculosis infected subject and a symptomatic M. paratuberculosis infected subject comprising the steps of: (a) selecting a group of miRNA molecules the differential expression of which may be associated with a disease's symptomatic state; (b) predicting the disease's symptomatic state; and (c) reducing the number of miRNAs in the panel to a minimum number to provide a panel of miRNAs that still produces a result.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate (one) several embodiment(s) of the invention and together with the description, serve to explain the principles of the invention.

FIG. 1 shows an example of expression analysis of the miRNA profiles for all samples.

FIG. 2 is a principal component analysis (PCA) showing test data analysis based on the first and second PCs (labelled Dim1 and Dim2 on the axis labels)

FIG. 3 shows marker ranking analysis of miRNA signal importance in the classification of samples according to a resampling procedure based on random permutations of the signals.

### DETAILED DESCRIPTION

The present invention may be understood more readily by reference to the following detailed description of preferred embodiments of the invention and the Examples included therein and to the Figures and their previous and following description.

### I. Definitions

To facilitate an understanding of the principles and features of the various embodiments of the disclosure, various illustrative embodiments are explained herein. Although exemplary embodiments of the disclosure are explained in detail, it is to be understood that other embodiments are contemplated. Accordingly, it is not intended that the disclosure is limited in its scope to the details of construction and arrangement of components set forth in the description or examples. The disclosure is capable of other embodiments and of being practiced or carried out in various ways.

In describing the exemplary embodiments, specific terminology will be resorted to for the sake of clarity. As used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value.

Similarly, as used herein, “substantially free” of something, or “substantially pure”, and like characterizations, can include both being “at least substantially free” of something, or “at least substantially pure”, and being “completely free” of something, or “completely pure.”

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

The phrase “nucleic acid” or “polynucleotide sequence” refers to a single or double-stranded polymer of deoxyribonucleotide or ribonucleotide bases read from the 5′ to the 3′ end. Nucleic acids may also include modified nucleotides that permit correct read-through by a polymerase and do not alter expression of a polypeptide encoded by that nucleic acid.

A “coding sequence” or “coding region” refers to a nucleic acid molecule having sequence information necessary to produce a gene product, when the sequence is expressed.

A “probe” is defined as a nucleic acid capable of binding to a target nucleic acid of complementary sequence through one or more types of chemical bonds, usually through complementary base pairing, usually through hydrogen bond formation. A probe may include natural (i.e., A, G, C, T or U) or modified bases (7-deazaguanosine, inosine, etc.). In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. Probes may bind target sequences lacking complete complementarity with the probe sequence depending upon the stringency of the hybridization conditions. The probes are preferably directly labeled as with isotopes, chromophores, lumiphores, chromogens, or indirectly labeled such as with biotin to which a streptavidin complex may later bind. By assaying for the presence or absence of the probe, one can detect the presence or absence of the select sequence or subsequence.

As used herein, the term “microRNA” or “miRNA” or “miR” designates a non-coding RNA molecule having a length of about 17 to 25 nucleotides, specifically having a length of 17, 18, 19, 20, 21, 22, 23, 24 or 25 nucleotides which hybridizes to and regulates the expression of a coding messenger RNA.

The term “miRNA molecule” refers to any nucleic acid molecule representing the miRNA, including natural miRNA molecules, i.e. the mature miRNA, pre-miRNA, pri-miRNA.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. In particular, an isolated nucleic acid of the present invention is separated from open reading frames that flank the desired gene and encode proteins other than the desired protein. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

The term “sample” generally refers to tissue or organ sample, blood, cell-free blood such as serum and plasma, urine, saliva, milk and cerebrospinal fluid sample.

As used herein, the term “blood sample” refers to serum, plasma, cell-free blood, whole blood and its components, blood derived products or preparations. Plasma and serum are very useful as shown in the examples. Specifically, the blood sample is a cell-free blood sample.

The term “quantifying” or “quantification” as used herein refers to absolute quantification, i.e. determining the amount of the respective miRNA but also encompasses measuring the level of the respective miRNA and comparing said level with reference or control miRNA, or comparative expression to other quantified miRNA. Quantification of the respective miRNA as listed in the tables herein allow expression profiling of samples and thus allow identification of signatures associated with diseased samples, as well as identification of signatures associated with prognosis and response to treatment. The quantity of miRNAs or difference in miRNA levels can be determined by any of the methods described herein.

A “control”, “control sample”, or “reference value” or “reference level” are terms which can be used interchangeably herein, and are to be understood as a sample or standard used for comparison with the experimental sample. The control may include a sample obtained from a healthy subject or a subject, which is not at risk of or suffering from Johne's Disease. Reference level specifically refers to the level of miRNA or miRNA expression quantified in a sample from a healthy subject, from a subject, which is not at risk of or suffering from Johne's Disease. Specifically a more than 1.3, 1.4, 1.5, 1.6, 1.7, 1.8, 1.9 or 2.0 fold difference between the reference level of one or more miRNAs as defined herein obtained from a sample of a subject. Additionally, a control may also be a standard reference value or range of values, i.e. such as stable expressed miRNAs in the samples, for example the endogenous control.

### II. Compositions

The present invention provides genomic identifiers for mycobacterial species. These can be used as target nucleic acid sequences for diagnosis of mycobacterial infection. The diagnostic targets can be used for identification of Johne's disease or infection in a sample.

The practice of the present invention employs, unless otherwise indicated, conventional techniques of molecular biology (including recombinant techniques), microbiology, cell biology, biochemistry, immunology, protein kinetics, and mass spectroscopy, which are within the skill of art. Such techniques are explained fully in the literature, such as Sambrook et al., 2000, Molecular Cloning: A Laboratory Manual, third edition, Cold Spring Harbor

Laboratory Press; Current Protocols in Molecular Biology Volumes 1-3, John Wiley & Sons, Inc.; Kriegler, 1990, Gene Transfer and Expression: A Laboratory Manual, Stockton Press, New York; Dieffenbach et al., 1995, PCR Primer: A Laboratory Manual, Cold Spring Harbor Laboratory Press, each of which is incorporated herein by reference in its entirety. Procedures employing commercially available assay kits and reagents typically are used according to manufacturer-defined protocols unless otherwise noted.

Generally, the nomenclature and the laboratory procedures in recombinant DNA technology described below are those well-known and commonly employed in the art. Standard techniques are used for cloning, DNA and RNA isolation, amplification and purification. Generally enzymatic reactions involving DNA ligase, DNA polymerase, restriction endonucleases and the like are performed according to the manufacturer's specifications.

Provided herein is a method for detecting the presence of Johne's disease or infection in a subject, comprising the steps of: (a) determining the level of expression of each of a plurality of miRNAs within a sample from a subject; and (b) using one or more Artificial Intelligence (AI) model to predict the disease condition of the subject.

A. miRNAs

Nucleotide sequences of mature miRNAs and their respective precursors are known in the art and available from the database miRBase or from Sanger database.

Identical polynucleotides as used herein in the context of a polynucleotide to be detected by the method as described herein may have a nucleic acid sequence with an identity of at least 90%, 95%, 97%, 98% or 99% or less than 3 or 2 single nucleotide modifications compared to a polynucleotide comprising or consisting of the nucleotide sequence of any one of SEQ ID NOs: 1-25.

Furthermore, identical polynucleotides as used herein in the context of a polynucleotide to be detected by the method as described herein may have a nucleic acid sequence with an identity of at least 90%, 95%, 97%, 98% or 99% to a polynucleotide comprising or consisting of the nucleotide sequence of any one of SEQ ID NOs: 1-25 including one, two, three or more nucleotides of the corresponding pre-miRNA sequence at the 5′end and/or the 3′end of the respective seed sequence.

All of the specified miRNAs used according to the invention also encompass isoforms and variants thereof. For the purpose of the invention, the terms “isoforms and variants” (which have also be termed “isomirs”) of a reference miRNA include trimming variants (5′ trimming variants in which the 5′ dicing site is upstream or downstream from the reference miRNA sequence; 3′ trimming variants: the 3′ dicing site is upstream or downstream from the reference miRNA sequence), or variants having one or more nucleotide modifications (3′ nucleotide addition to the 3′ end of the reference miRNA; nucleotide substitution by changing nucleotides from the miRNA precursor), or the complementary mature microRNA strand including its isoforms and variants (for example for a given 5′ mature microRNA the complementary 3′ mature microRNA and vice-versa). With regard to nucleotide modification, the nucleotides relevant for RNA/RNA binding, i.e. the 5′-seed region and nucleotides at the cleavage/anchor side are excluded from modification.

In the following, if not otherwise stated, the term “miRNA” encompasses 3p and 5p strands and also its isoforms and variants.

The plurality of miRNAs form a panel comprising the following: bta-miR-19b, bta-miR-196b, bta-miR-146a, bta-miR-29b, bta-miR-29a, bta-miR-142-3p, bta-miR-155, bta-miR-582, bta-miR-21-5p, bta-miR-6517, bta-miR-7857-5p, bta-miR-24, bta-miR-378c, bta-miR-1271, bta-miR-100, bta-miR-301a, bta-miR-32, bta-miR-1247-5p, bta-miR-184, bta-miR-202, bta-miR-137, bta-miR-105a, bta-miR-433, bta-miR-133b, bta-miR-93, bta-miR-20a-5p, bta-miR-16a-5p, bta-miR-17-5p, bta-miR-92a, oan-miR-7417-5p, cel-miR-70-3p and ath-miR-167d.

The names of the miRNA molecules and associated sequences that are used in the method of the invention are set out below in Table 1.

TABLE 1
miRNA Molecule Sequences

| | SEQ | |
|---|---|---|
| | ID | |
| miRNA Name | NO: | Sequence |
| bta-miR-19b | 1 | UGUGCAAAUCCAUGCAAAACUGA |
| bta-miR-196b | 2 | UAGGUAGUUUCCUGUUGUUGGGA |
| bta-miR-146a | 3 | UGAGAACUGAAUUCCAUAGGUUGU |
| bta-miR-29b | 4 | UAGCACCAUUUGAAAUCAGUGUU |
| bta-miR-29a | 5 | CUAGCACCAUCUGAAAUCGGUUA |
| bta-miR-142-3p | 6 | AGUGUUUCCUACUUUAUGGAUG |
| bta-miR-155 | 7 | UUAAUGCUAAUCGUGAUAGGGGU |
| bta-miR-582 | 8 | UUACAGUUGUUCAACCAGUUACU |
| bta-miR-21-5p | 9 | UAGCUUAUCAGACUGAUGUUGACU |
| bta-miR-6517 | 10 | UCAGGGUCCGUGAGCUCCUCGGC |
| bta-miR-7857-5p | 11 | AUAGCCAGUUGGGGAAGAAUGC |
| bta-miR-24 | 12 | GUGCCUACUGAGCUGAUAUCAGU |
| bta-miR-378c | 13 | ACUGGACUUGGAGUCAGAAGU |
| bta-miR-1271 | 14 | CUUGGCACCUAGUAAGUACUCA |
| bta-miR-100 | 15 | AACCCGUAGAUCCGAACUUGUG |
| bta-miR-301a | 16 | CAGUGCAAUAGUAUUGUCAAAGCAU |
| bta-miR-32 | 17 | UAUUGCACAUGACUAAGUUGCAU |
| bta-miR-1247-5p | 18 | ACCCGUCCCGUGCGUCCCCGGA |
| bta-miR-184 | 19 | UGGACGGAGAACUGAUAAGGGU |
| bta-miR-202 | 20 | UUCCUAUGCAUAUACUUCUUU |
| bta-miR-137 | 21 | UUAUUGCUUAAGAAUACGCGUAG |
| bta-miR-105a | 22 | UCAAAUGCUCAGACUCCUGUGGU |
| bta-miR-433 | 23 | AUCAUGAUGGGCUCCUCGGUGU |
| bta-miR-133b | 24 | UUUGGUCCCCUUCAACCAGCUA |
| bta-miR-20a-5p | 25 | UAAAGUGCUUAUAGUGCAGGUAG |

The method further comprises the use of at least one normalizer and/or an off-species control miRNA molecule. At least one normalizer is used to ‘normalize’ data, i.e. to control for variation between the samples tested in the method of the invention, and the at least one control is used to try to ensure there are no failure or false readings in the results. An off-species control is added in to show that the miRNAs detected are relevant to the species panel. The off-species control is an miRNA from another species, i.e. not a ruminant. Advantageously, the use of an off-species controls provides another layer of control to distinguish between background or non-specific signals and a positive result.

The sequences of the normalizers and the off-species controls that were used are provided below in Table 2.

TABLE 2
Normalizers and Off-species Controls

| | SEQ ID | |
|---|---|---|
| Normalizers | Number | Sequence |
| bta-miR-93 | 26 | CAAAGUGCUGUUCGUGCAGGUA |
| bta-miR-16a-5p | 27 | UAGCAGCACGUAAAUAUUGGUG |
| bta-miR-17-5p | 28 | CAAAGUGCUUACAGUGCAGGUAGU |
| bta-miR-92a | 29 | UAUUGCACUUGUCCCGGCCUGU |
| Off-species | | |
| controls | | |
| oan-miR-7417-5p | 30 | UUCCCCACUCUGAGCACACAGC |
| cel-miR-70-3p | 31 | UAAUACGUCGUUGGUGUUUCCAU |
| ath-miR-167d | 32 | UGAAGCUGCCAGCAUGAUCUGG |

It is preferred that the method comprises the step of assessing the relative levels of miRNA expression of each one of miRNA molecules bta-miR-19b, bta-miR-196b, bta-miR-146a, bta-miR-29b, bta-miR-29a, bta-miR-142-3p, bta-miR-155, bta-miR-582, bta-miR-21-5p, bta-miR-6517, bta-miR-7857-5p, bta-miR-24, bta-miR-378c, bta-miR-1271, bta-miR-100, bta-miR-301a, bta-miR-32, bta-miR-1247-5p, bta-miR-184, bta-miR-202, bta-miR-137, bta-miR-105a, bta-miR-433, bta-miR-133b, bta-miR-93, bta-miR-20a-5p, bta-miR-16a-5p, bta-miR-17-5p, bta-miR-92a, oan-miR-7417-5p, cel-miR-70-3p and ath-miR-167d within a sample from a subject and using the data obtained from measurement of the expression levels to determine the presence or absence of disease in a subject.

The plurality of target miRNAs form a panel comprising the following: bta-miR-19b, bta-miR-196b, bta-miR-146a, bta-miR-29b, bta-miR-29a, bta-miR-142-3p, bta-miR-155, bta-miR-582, bta-miR-21-5p, bta-miR-6517, bta-miR-7857-5p, bta-miR-24, bta-miR-378c, bta-miR-1271, bta-miR-100, bta-miR-301a, bta-miR-32, bta-miR-1247-5p, bta-miR-184, bta-miR-202, bta-miR-137, bta-miR-105a, bta-miR-433, bta-miR-133b, bta-miR-93, bta-miR-20a-5p, bta-miR-16a-5p, bta-miR-17-5p, bta-miR-92a, oan-miR-7417-5p, cel-miR-70-3p and ath-miR-167d.

### III. Methods of Detection

### A. Identification of Mycobacteruim

Provided herein are methods for detecting the presence of mycobacterial disease in a subject. The genus Mycobacterium includes pathogens known to cause serious diseases in mammals, including, for example, tuberculosis and leprosy. Mycobacterium (also referred to as mycobacteria) do not contain endospores or capsules, and are usually considered Gram-positive. In addition to the usual fatty acids found in membrane lipids, mycobacteria have a wide variety of very long-chain saturated (C₁₈-C₃₂) and monounsaturated (up to C₂₆) n-fatty acids. The occurrence of α-alkyl β-hydroxy very long-chain fatty acids, i.e., mycolic acids, is a hallmark of mycobacteria and related species. Mycobacterial mycolic acids are large (C₇₀-C₉₀) with a large a-branch (C₂₀-C₂₅). The main chain contains one or two double bonds, cyclopropane rings, epoxy groups, methoxy groups, keto groups or methyl branches. Such acids are major components of the cell wall, occurring mostly esterified in clusters of four on the terminal hexa-arabinofuranosyl units of the major cell-wall polysaccharides called arabinogalactans. They are also found esterified to the 6 and 6′ positions of trehalose to form ‘cord factor’. Small amounts of mycolate are also found esterified to glycerol or sugars such as trehalose, glucose and fructose depending on the sugars present in the culture medium. Mycobacteria also contain a wide variety of methyl-branched fatty acids. These include 10-methyl C₁₈ fatty acid (tuberculostearic acid found esterified in phosphatidyl inositide mannosides), 2,4-dimethyl C₁₄ acid and mono-, di- and trimethyl-branched C₁₄ to C₂₅ fatty acids found in trehalose-containing lipooligosaccharides, trimethyl unsaturated C₂₇ acid (phthienoic acid), tetra-methyl-branched C₂₈-C₃₂ faccy acids (mycocerosic acids) and shorter homologues found in phenolic glycolipids and phthiocerol esters, and multiple methyl-branched phthio-ceranic acids such as hepamethyl-branched C₃₇ acid and oxygenated multiple methyl-branched acids such as 17-hydroxy-2,4,6,8,10,12,14,16-octamethyl C₄₀ acid found in sulpholipids. In addition, mycocerosic acids and other branched acids are esterified to phthicerol and phenolphthicerol and their derivates. Kolattukudy et al., Mol. Microbio. 24(2):263-270 (1997). Evidence implicates specific cell envelope lipids in Mtb pathogenesis. Rao, et al., J. Exp. Med., 201(4):535-543 (2005).

Mycobacterium species include, but are not limited to: M. abscessus; M. africanum; M. agri; M. aichiense; M. alvei; M. arupense; M. asiaticum; M. aubagnense; M. aurum; M austroafricanum; Mycobacterium avium complex (MAC); M. avium; M. avium paratuberculosis, which has been implicated in Crohn's disease in humans and Johne's disease in cattle and sheep; M. avium silvaticum; M. avium “hominissuis”; M. colombiense; M. boeitickei; M. bohemicum; M. bolletii; M. botniense; M. bovis; M. branderi; M. brisbanense; M. brumae; M. canariasense; M. caprae; M. celatum; M. chelonae; M. chimaera; M. chitae; M. chlorophenolicum; M. chubuense; M. conceptionense; M. confluentis; M. conspicuum; M. cookii; M. cosmeticum; M. diernhoferi; M. doricum; M. duvalii; M. elephantis; M. fallax; M. farcinogenes; M. flavescens; M. florentinum; M. fluoroanthenivorans; M. fortuitum; M. fortuitum subsp. acetamidolyticum; M. frederiksbergense; M. gadium; M. gastri; M. genavense; M. gilvum; M. goodii; M. gordonae; M. haemophilusassiacum; M. heckeshornense; M. heidelbergense; M. hiberniae; M. hodleri; M. holsaticum; M. houstonense; M. immunogenum; M. interjectum; M. intermedium; M. intracellulare; M. kansasii; M. komossense; M. kubicae; M. kumamotonense; M. lacus; M. lentiflavum; M. leprae, which causes leprosy; M. lepraemurium; M. madagascariense; M. mageritense; M. malmoense; M. marinum; M. massiliense; M. microti; M. monacense; M. montefiorense; M. moriokaense; M. mucogenicum; M. murale; M. nebraskense; M. neoaurum; M. neworleansense; M. nonchromogenicum; M. novocastrense; M. ohuense; M. palustre; M. parafortuitum; M. parascrofulaceum; M. parmense; M. peregrinum; M. phlei; M phocaicum; M. pinnipedii; M. porcinum; M. poriferae; M. pseudoshottsii; M. pulveris; M. psychrotolerans; M. pyrenivorans; M. rhodesiae; M. saskatchewanense; M. scrofidaceum; M. senegalense; M. seoulense; M. septicum; M. shimoidei; M. shottsii; M. simiae; M. smegmatis; M. sphagni; M. szulgai; M. terrae; M. thermoresistibile; M. tokaiense; M. triplex; M. triviale; Mycobacterium tuberculosis complex (MTBC), members are causative agents of human and animal tuberculosis (M. tuberculosis, the major cause of human tuberculosis; M. bovis; M. bovis BCG; M. africanum; M. canetti; M. caprae; M. pinnipedii'); M. tusciae; M. ulcerans, which causes the “Buruli”, or “Bairnsdale, ulcer”; M. vaccae; M. vanbaalenii; M. wolinskyi; and M. xenopi.

Mycobacteria can be classified into several groups for purpose of diagnosis and treatment, for example: M. tuberculosis complex (MTB) which can cause tuberculosis: M. tuberculosis, M. africanum, M. bovis, M. bovis BCG, M. caprae, M. microti, M. pinnipedii, the dassie bacillus, and M. canettii (proposed name) (Somoskovi, et al., J. Clinical Microbio 45(2):595-599 (2007)); M. leprae which causes Hansen's disease or leprosy; nontuberculous mycobacteria (NTM) are all the other mycobacteria which can cause pulmonary disease resembling tuberculosis, lymphadenitis, skin disease, or disseminated disease. MTB members show a high degree of genetic homogeneity (Somoskovi et al, 2007). The mycobacteria of the invention is selected from a group consisting of mycobacterial disease, including but not restricted to Johne's disease, tuberculosis and Crohn's disease, or any other disease caused by Mycobacterium avium subspecies paratuberculosis (MAP) or any other species within the Mycobacterium genus.

### 1. Johne's Disease

Provided herein are methods of detecting mycobacterial disease, including Johne's disease. Mycobacterium paratuberculosis causes Johne's disease (paratuberculosis) in dairy cattle. The disease is characterized by chronic diarrhea, weight loss, and malnutrition, resulting in estimated losses of $220 million per year in the USA alone with up to 50% of the herds in some areas within the USA (Wisconsin and Alabama). Cows infected with

Johne's disease are known to excrete Mycobacterium paratuberculosis in their milk and feces. In humans, M. paratuberculosis bacilli have been found in tissues examined from Crohn's disease patients indicating possible zoonotic transmission from infected dairy products to humans. Unfortunately, the virulence mechanisms controlling M. paratuberculosis persistence inside the host are poorly understood, and the key steps for establishing the presence of paratuberculosis are elusive. Mechanisms responsible for invasion and persistence of M. paratuberculosis inside the intestine remain undefined on a molecular level (Valentin-Weigand & Goethe, 1999). Both live and dead bacilli are observed in sub-epithelial macrophages after uptake. Once inside the macrophages, M. paratuberculosis survive and proliferate inside the phagosomes using unknown mechanisms.

M. paratuberculosis is closely related to Mycobacterium avium subspecies avium (hereinafter referred to as Mycobacterium avium or M. avium), which is a persistent health problem for immunocompromised humans, particularly HIV-positive individuals. Limited tools are available to researchers to definitively identify M. paratuberculosis and to distinguish it from M. avium. Existing methods are subject to high cross-reactivity, poor sensitivity, specificity, and predictive value. This dearth of knowledge translates into a lack of suitable vaccines for prevention and treatment of Johne's disease in animals, and of Crohn's disease in humans.

Many clinical methods for detecting and identifying Mycobacterium species in samples require analysis of the bacterium's physical characteristics (e.g., acid-fast staining and microscopic detection of bacilli), physiological characteristics (e.g., growth on defined media) or biochemical characteristics (e.g., membrane lipid composition). These methods require relatively high concentrations of bacteria in the sample to be detected, may be subjective depending on the clinical technician's experience and expertise, and are time-consuming.

Because Mycobacterium species are often difficult to grow in vitro and may take weeks to reach a useful density in culture, these methods can also result in delayed patient treatment and costs associated with isolating an infected individual until the diagnosis is completed.

Provided herein are methods of selecting a panel for use in disease diagnosis comprising the steps of: selecting a group of miRNA molecules the differential expression of which may be associated with a disease condition; training one or more AI model to be able to predict the disease condition; and using the one or more AI model to reduce the number of miRNAs in the panel to a minimum number to provide a panel of miRNAs that still produces a result. Also provided herein are methods of selecting a nucleic acid panel for use in differential diagnosis between a symptom-free M. paratuberculosis infected subject and a symptomatic M. paratuberculosis infected subject. Further provided are methods of selecting a panel for use in predicting whether the subject will exhibit disease symptoms after mycobacterial infection.

More recently, assays that detect the presence of nucleic acid derived from bacteria in the sample have been preferred because of the sensitivity and relative speed of the assays. In particular, assays that use in vitro nucleic acid amplification of nucleic acids present in a clinical sample can provide increased sensitivity and specificity of detection. Such assays, however, can be limited to detecting one or a few Mycobacterium species depending on the sequences amplified and/or detected.

### B. Identification of Target Sequences

The present invention relates to a method for detecting the presence or amount of a target polynucleotide (nucleic acid sequence) from the host's response to Mycobacterium paratuberculosis in a sample. The target polynucleotide is a virulence determinant. In a preferred embodiment, the target polynucleotide is a miRNA. The invention is also directed to a method of detecting the presence of a disease or infection state in a mammal, by detecting the presence or amount of a target miRNA, wherein the presence or amount of the target miRNA identifies the disease state. Thus, the invention relates to diagnostic compositions and methods for detecting Johne's disease. The sample containing the target miRNA may be tissue, collection of cells, cell lysate, body fluid, excretum, in vitro culture, purified polynucleotide, isolated polynucleotide, food sample, medical sample, agro-livestock sample, or environmental sample.

The invention described here utilizes large-scale identification of disrupted genes and the use of bioinformatics and AI to select mutants that could be characterized in animals.

C. Multiplex miRNA Profiling

The present invention uses multiplex miRNA profiling without RNA purification. Accuracy of miRNA profiling is enhanced when sample processing can be kept to a minimum, avoiding steps such as RNA purification that can introduce bias and inaccuracies. The present invention used a multiplex circulating miRNA assay that enables the profiling of a plurality of miRNAs in the same well directly from the sample, with no need for RNA purification. In one embodiment, the assay uses FirePlex® particles, which enable the multiplex capture of miRNAs with picomolar sensitivity and high specificity. The FirePlex® particles contain three distinct functional regions that are separated from each other by inert spacer regions. The central region of each particle is known as a central analyte or miRNA quantification region which contains miRNA probes that can capture target miRNAs. The central region of the particle comprises a reporter dye. The two end regions of each particle act as two halves of a barcode that distinguish between different particles. Detection is carried out using a flow cytometer to detect miRNA molecules that emit fluorescence that is proportional to their abundance in the sample. The flow cytometer was used to detect the fluorescence signal from the center of each particle through the reporter dye. Each miRNA that was used was given a unique code (up to 70 different codes were possible). The data that was obtained from the mixture of particles could then be attributed to the miRNAs by identification of the code.

The disease is selected from the group consisting of mycobacterial disease and related conditions.

The sample is a biofluid selected from the group consisting of blood, urine, milk, tissue fluid, saliva, cerebrospinal fluid (CSF) or another biofluid.

From the results of the experiments below, a differentiation in expression levels of miRNA was identified when comparing healthy animals with animals that have mycobacterial disease.

### D. Predictive Modelling

Provided herein are methods using predictive modelling to investigate the scope to use the miRNA profiles to predict the presence or absence of disease. A group of healthy and unhealthy animals were taken and tested to determine the level of miRNA expression in samples from these animals. The data obtained was then used to train the models.

Fifteen machine learning models were fitted and compared with the aim of obtaining the best predictions of the disease outcome. Formal assessment of performance was conducted by computing a number of performance statistics based on 5-time repeated 10-fold cross-validation. Cross-validation was useful to obtain more realistic model performance measures from the training data.

Data from the FirePlex® analysis from each of the twenty five miRNA molecules from Table 1 was fitted to each of the models.

### E. Kits

Also provided herein is a kit for use in performing the method of the first aspect comprising means for determining the level of expression of each one of the following miRNA molecules: bta-miR-19b, bta-miR-196b, bta-miR-146a, bta-miR-29b, bta-miR-29a, bta-miR-142-3p, bta-miR-155, bta-miR-582, bta-miR-21-5p, bta-miR-6517, bta-miR-7857-5p, bta-miR-24, bta-miR-378c, bta-miR-1271, bta-miR-100, bta-miR-301a, bta-miR-32, bta-miR-1247-5p, bta-miR-184, bta-miR-202, bta-miR-137, bta-miR-105a, bta-miR-433, bta-miR-133b, bta-miR-93, bta-miR-20a-5p, bta-miR-16a-5p, bta-miR-17-5p, bta-miR-92a, oan-miR-7417-5p, cel-miR-70-3p and ath-miR-167d.

There is also provided a method of selecting a panel for use in disease diagnosis comprising the steps of: (a) selecting a group of miRNA molecules the differential expression of which may be associated with a disease condition; (b) training one or more AI model to be able to predict the disease condition; and (c) using the one or more AI model to reduce the number of miRNAs in the panel to a minimum number to provide a panel of miRNAs that still produces a result

### IV. Exemplary Embodiments

Exemplary Embodiments of the present disclosure include, but are not limited to,

the following.

Embodiment 1: A method for detecting the presence of mycobacterial disease or infection in a subject, comprising the steps of: (a) determining a level of expression of each of a plurality of miRNA molecules within a sample from a subject; and (b) using one or more Artificial Intelligence (AI) model to predict the disease condition of the subject.

Embodiment 2: The method according to Embodiment 1, wherein the one or more AI model compares the level of expression of each miRNA molecule with at least one pre-determined reference level characteristic of a non-diseased subject for each one of the plurality of the miRNA molecules of step (a), wherein a deviation of the level of expression of said miRNA molecules from step (a) in comparison with the at least one reference level allows for the diagnosis and/or prognosis of the disease.

Embodiment 3: The method according to Embodiment 1, wherein the plurality of miRNA molecules is selected from a group consisting of bta-miR-19b, bta-miR-196b, bta-miR-146a, bta-miR-29b, bta-miR-29a, bta-miR-142-3p, bta-miR-155, bta-miR-582, bta-miR-21-5p, bta-miR-6517, bta-miR-7857-5p, bta-miR-24, bta-miR-378c, bta-miR-1271, bta-miR-100, bta-miR-301a, bta-miR-32, bta-miR-1247-5p, bta-miR-184, bta-miR-202, bta-miR-137, bta-miR-105a, bta-miR-433, bta-miR-133b, bta-miR-93, bta-miR-20a-5p, bta-miR-16a-5p, bta-miR-17-5p and bta-miR-92a; with oan-miR-7417-5p, cel-miR-70-3p and ath-miR-167d as off-species controls.

Embodiment 4: The method according to Embodiment 1, wherein the subject is a mammal.

Embodiment 5: The method according to Embodiment 4, wherein the subject is a cow, sheep, goat, deer, llama, alpaca or vicuna.

Embodiment 6: The method according to Embodiment 1, wherein the method further comprises the step of using a machine learning algorithm for predictive modelling.

Embodiment 7: The method according to Embodiment 1, wherein the method comprises the use of a combination of AI models.

Embodiment 8: The method according to Embodiment 1, wherein the method further comprises the use of at least one normalizer and/or control miRNA molecule.

Embodiment 9: The method according to Embodiment 8, wherein the control miRNA molecule is an off-species control miRNA molecule.

Embodiment 10: The method according to Embodiment 8, wherein the at least one normalizer is selected from a group consisting of bta-miR-93, bta-miR-16a-5p, bta-miR-17-5p and/or bta-miR-92a.

Embodiment 11: The method according to any one of Embodiment 9, wherein the at least one off-species control is selected from a group consisting of oan-miR-7417-5p, cel-miR-70-3p and/or ath-miR-167d.

Embodiment 12: The method according to Embodiment 1, wherein the disease is selected from a group consisting of mycobacterial disease, including but not restricted to Johne's disease, tuberculosis and Crohn's disease, or any other disease caused by Mycobacterium aviumsubspecies paratuberculosis (MAP) or any other species within the Mycobacterium genus.

Embodiment 13: The method according to Embodiment 1, wherein the sample is a biofluid selected from the group consisting of blood, urine, milk, tissue fluid, saliva, cerebrospinal fluid (CSF), feces or another biofluid.

Embodiment 14: The method according to Embodiment 1, wherein the miRNAs are cell free miRNAs.

Embodiment 15: A kit for use in performing the method of Embodiment 1 comprising means for determining the level of expression of each one of the following miRNA molecules: bta-miR-19b, bta-miR-196b, bta-miR-146a, bta-miR-29b, bta-miR-29a, bta-miR-142-3p, bta-miR-155, bta-miR-582, bta-miR-21-5p, bta-miR-6517, bta-miR-7857-5p, bta-miR-24, bta-miR-378c, bta-miR-1271, bta-miR-100, bta-miR-301a, bta-miR-32, bta-miR-1247-5p, bta-miR-184, bta-miR-202, bta-miR-137, bta-miR-105a, bta-miR-433, bta-miR-133b, bta-miR-93, bta-miR-20a-5p, bta-miR-16a-5p, bta-miR-17-5p, bta-miR-92a, oan-miR-7417-5p, cel-miR-70-3p and ath-miR-167d.

Embodiment 16: A method of selecting a panel for use in disease diagnosis comprising the steps of: (a) selecting a group of miRNA molecules the differential expression of which may be associated with a disease condition; (b) training one or more AI model to be able to predict the disease condition; and (c) using the one or more AI model to reduce the number of miRNAs in the panel to a minimum number to provide a panel of miRNAs that still produces a result.

Embodiment 17: A method of selecting a panel for use in differential diagnosis between a symptom-free M. paratuberculosis infected subject and a symptomatic M. paratuberculosis infected subject comprising the steps of: selecting a group of miRNA molecules the differential expression of which may be associated with a disease's symptomatic state; training one or more AI model to be able to predict the disease's symptomatic state; and using the one or more AI model to reduce the number of miRNAs in the panel to a minimum number to provide a panel of miRNAs that still produces a result.

Embodiment 18: A method of selecting a panel for use in predicting whether the subject will exhibit disease symptoms after mycobacterial infection comprising the steps of: selecting a group of miRNA molecules the differential expression of which may be associated with an exhibition of disease symptoms; training one or more AI model to be able to predict the exhibition of symptoms; and using the one or more AI model to reduce the number of miRNAs in the panel to a minimum number to provide a panel of miRNAs that still produces a result.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Unless otherwise indicated, all numbers expressing quantities of components, molecular weights, formula weights and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

For any method disclosed herein that includes discrete steps, the steps may be conducted in any feasible order. And, as appropriate, any combination of two or more steps may be conducted simultaneously.

The description exemplifies illustrative embodiments. In several places throughout the application, guidance is provided through lists of examples, which examples can be used in various combinations. In each instance, the recited list serves only as a representative group and should not be interpreted as an exclusive list.

All headings are for the convenience of the reader and should not be used to limit the meaning of the text that follows the heading, unless so specified.

The present invention is illustrated by the following examples. It is to be understood that the particular examples, materials, amounts, and procedures are to be interpreted broadly in accordance with the scope and spirit of the invention as set forth herein.

### EXAMPLES

### Example 1: miRNA Assay to Accurately Identify the Presence or Absence of Mycobacterial Disease

### Materials and Methods

Samples were obtained from diseased and healthy cows. Diseased animals were selected on the basis of their positivity to Johne's disease on ELISA antibody testing.

A particle mixture was added to each well of a 96 well microtiter plate. The particle mixture contained around 32 particles that are specific for miRNA molecules. The particle mixture was suspended in 10 μl biofluid taken from cow subjects. In this case, the biofluid was blood. The particles were passed through a flow cytometer and around 20 readings were obtained for each of the 32 miRNA molecules from Table 1 and Table 2.

The above method was carried out using FirePlex® Particle Technology (Abcam). FirePlex® Particle Technology uses FirePlexR particles (Abcam) which are made from a porous bio-inert hydrogel that allows targets to be captured throughout a 3D volume. The FirePlex® assay protocol that was used in this example can be found in the FirePlexR miRNA Assay V3-Assay Protocol (Protocol Booklet Version 2.0, September 2018).

After the data acquisition, FirePlex® Analysis Workbench software was used to merge the events that were obtained from the three regions of the particles into a single event. Abundance data was then obtained for each miRNA molecule.

### Results

The data set for this experiment included 131 miRNA samples. The data set included 66 diseased and 65 control samples.

An example of the data obtained from the above experiment is provided below in Table 3. As mentioned above, the data set included 131 miRNA samples. The results below are shown for one of the diseased samples and one of the control samples used in this experiment. Data was collected for each of the 25 miRNA samples mentioned in Table 1. The results obtained with the seven normalizers and off-species controls as mentioned in Table 2 are also shown.

TABLE 3
miRNA Data Set

| | | bta-miR- | bta-miR- | bta-miR- | bta-miR- | bta-miR- | bta-miR- | bta-miR- | bta-miR- |
|---|---|---|---|---|---|---|---|---|---|
| Species | Diagnosis | 19b | 196b | 146a | 29b | 29a | 142 3p | 155 | 582 |
| Bovine | diseased | 315948.4 | 9512.912 | 13065.09 | 196450.1 | 118739.3 | 32433.99 | 8629.473 | 6619.878 |
| Bovine | control | 231121 | 37061.43 | 20158.24 | 498660 | 331491.9 | 52719.45 | 4975.901 | 4132.603 |
| | | bta-miR- | bta-miR- | bta-miR- | bta-miR- | bta-miR- | bta-miR- | bta-miR- | bta-miR- |
| Species | Diagnosis | 21-5p | 6517 | 7857-5p | 24 | 378c | 1271 | 100 | 20a-5p |
| Bovine | diseased | 910755.5 | 7907.182 | 13849.39 | 10451.63 | 50080.33 | 9274.427 | 21851.83 | 1277551 |
| Bovine | control | 531255 | 26031.37 | 28806.72 | 41511.4 | 37340.16 | 24189.02 | 111661.9 | 783108.1 |
| | | bta-miR- | bta-miR- | bta-miR- | bta-miR- | bta-miR- | bta-miR- | bta-miR- | bta-miR- |
| Species | Diagnosis | 32 | 1247-5p | 184 | 202 | 137 | 105a | 433 | 133b |
| Bovine | diseased | 12004.64 | 9384.475 | 17322.84 | 7107.03 | 6878.594 | 15421.65 | 193967.4 | 17149.18 |
| Bovine | control | 9710.609 | 8651.337 | 68454.24 | 8581.526 | 7020.674 | 50533.59 | 282891.3 | 21115.73 |

| | | bta-miR-93 | bta-miR-16a-5p |
|---|---|---|---|
| Species | Diagnosis | Normalizer | Normalizer |
| Bovine | diseased | 1244041 | 986831.4 |
| Bovine | control | 753095.1 | 984772.3 |

| | | bta-miR-17-5p | bta-miR-92a |
|---|---|---|---|
| Species | Diagnosis | Normalizer | Normalizer |
| Bovine | diseased | 1437131 | 261721.1 |
| Bovine | control | 880032.3 | 176453.1 |

Along with the above, pre-processed miRNA profiles consisting of 32 signals were provided for each sample. The objective was to to investigate differences between groups according to miRNA profiles and to build a predictive model of disease outcome based on the miRNA signals. The predictive model assessed the ability of miRNA signals to discriminate between groups and produce a reliable prediction of the class of a sample, identifying the most relevant signals in doing so.

### Exploratory Data Analysis

Exploratory Data Analysis was carried out to examine data and look for trends of the results following the FirePlex® analysis.

The Exploratory Data Analysis was carried out for information purposes, e.g. to understand any trends that were seen in the data.

Some pre-processing was conducted and two samples, one from each group, had to be discarded during pre-processing due to poor signal quality. The signals were log-transformed for improved visualization.

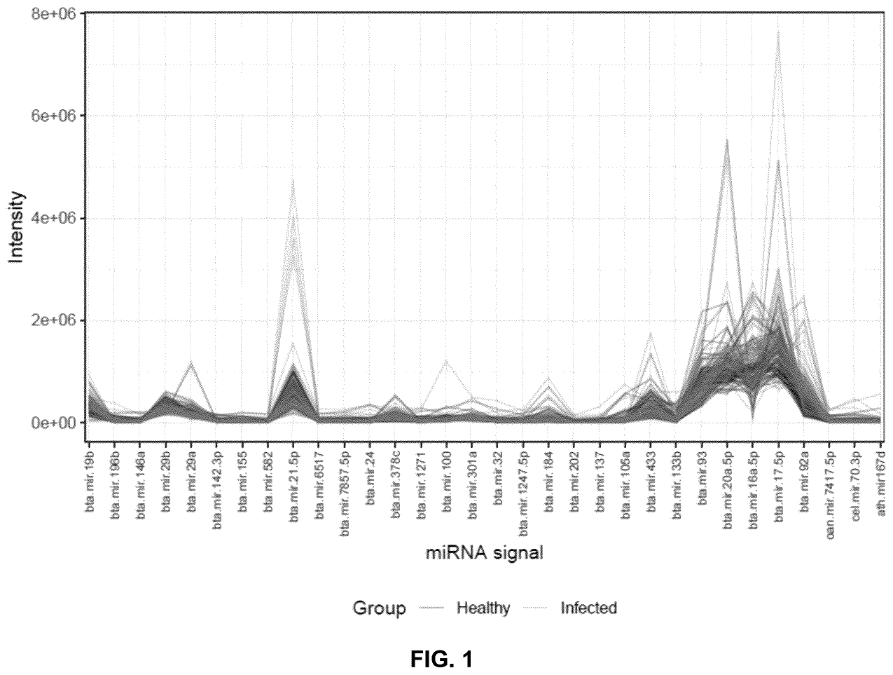

FIG. 1 shows the miRNA profiles for all samples. They are colored according to sample group. From left to right, the most distinguishable signals were bta-miR-21-5p, being particularly higher for some samples in the diseased samples, and bta-miR-20a-5p and bta-miR-17-5p, being variably high in both healthy and diseased samples. The highest variability between samples is observed for these two latter signals. In the middle section of the miRNA profiles, healthy signals seem to tend to have larger intensities except for a few outlying infected signals.

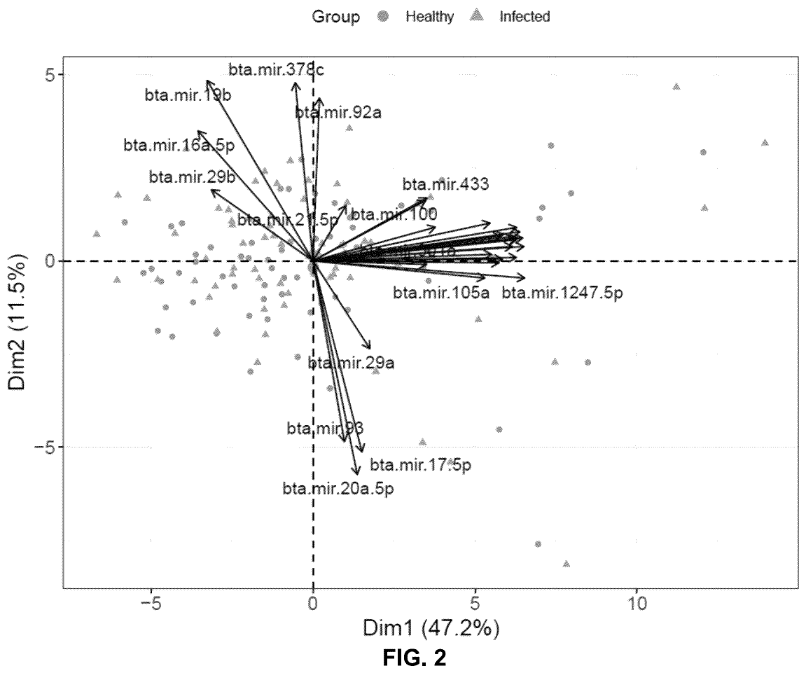

Principal component analysis (PCA) was used to represent the data set in low-dimensions so that potential patterns can be further explored. A PCA biplot jointly represents both samples and miRNA signals, using point and rays respectively. The proximity between points relates to the similarity between samples according to their miRNA profiles. The rays indicate directions of increasing intensity of the signals, whereas the angles between rays are related to the correlations between them: the smaller the angle the higher the positive correlation, the closer to right angle the weaker the correlation, and the closer to straight angle the higher the negative correlation. The biplot shown in FIG. 2 is based on the first and second PCs (labelled Dim1 and Dim2 on the axis labels) is shown in the following figure (the signals were log-transformed to facilitate visualisation). This biplot accounts for 58.63% of the total original data variability (contribution of each PC shown on the axis labels). Hence, for the present purposes, a PCA biplot facilitates the visualisation and identification of patterns in the data.

### Predictive Modelling

The objective of the predictive modelling was to investigate the scope to use the miRNA profiles to predict the presence or absence of disease.

A group of healthy and unhealthy animals were taken and tested to determine the level of miRNA expression in samples from these animals. The data obtained was then used to train the models.

Fifteen machine learning models were fitted and compared with the aim of obtaining the best predictions of the disease outcome. A number of statistics based on 5-time repeated 10-fold cross-validation were calculated for each model. Cross-validation was useful to obtain more realistic model performance measures from the training data.

Data from the FirePlex® analysis from each of the thirty-two miRNA molecules from Table 1 and Table 2 was fitted to each of the models.

No notable differences were found between models.

Table 4 shows the performance measure from the top performing model. The first section shows a cross-validated confusion matrix comparing predicted with actual group. The overall percentual errors for each group are reported off the diagonal. The highest overall error is 15.73%, corresponding to infected samples being wrongly classed as healthy. The cross-validated model accuracy is 71.76% (95% confidence interval: [0.6814, 0.7518]). This is significantly over the no information rate (p<0:0001), meaning that the model statistically performs better than random allocation. This is supported by the Kappa statistics which indicates how good the prediction is in relation to random allocating of samples into groups. This is also supported by the values of sensitivity and specificity of the test, particularly in terms of the ability to rightly identify healthy samples: 68.79% of infected samples were classed as infected whereas 74.77% healthy samples were classed as healthy.

TABLE 4
Confusion Matrix and Statistics

| Reference | | |
|---|---|---|
| Prediction | Diseased | Control |
| Diseased | 0.3466 | 0.1252 |
| Control | 0.1573 | 0.3710 |
| | Accuracy: | 0.7176 |
| | 95% CI: | (06814, 0.7518) |
| | No Information Rate | 0.5038 |
| | P-Value [Acc > NIR]: | <2e−16 |
| | Kappa: | 0.4354 |
| | Mcnemar's Test P-Value: | 0.1414 |
| | Sensitivity: | 0.6879 |
| | Specificity: | 0.7477 |
| | Pos Pred Value: | 0.7346 |
| | Neg Pred Value: | 0.7023 |
| | Prevalence: | 0.5038 |
| | Detection Rate: | 0.3466 |
| | Detection Prevalence: | 0.4718 |
| | Balanced Accuracy: | 0.7178 |
| | ‘Positive’ Class | Diseased |

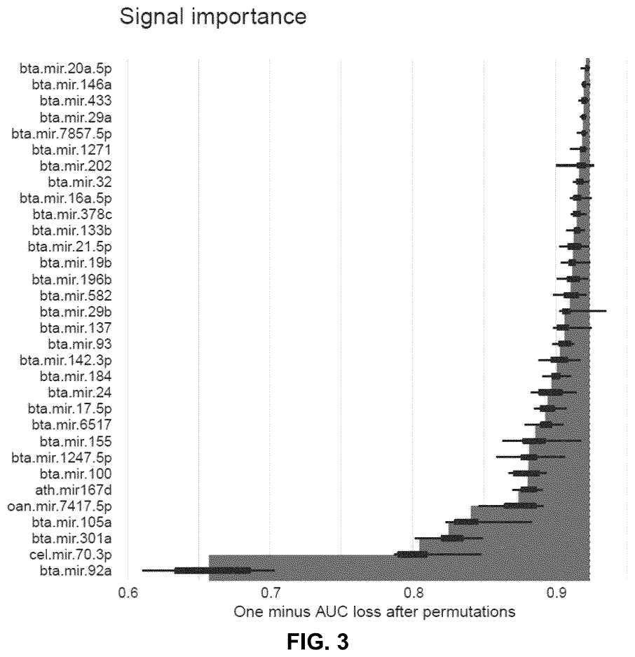

FIG. 3 shows estimates of miRNA signal importance in the classification of samples according to a resampling procedure based on random permutations of the signals. The signals are ranked from top to bottom according to their importance.

According to these results, the top 5 signals in terms of discriminating power are bta.miR.20a.5p, bta.miR.433, bta.miR.29a, bta.miR.7857.5p and bta.miR.146a.

From the results presented herein, it can be seen that the predictive models based on miRNA data are able to differentiate between control and diseased samples with around 72% accuracy in samples. Test sensitivity and specificity were also similar.

From the results presented herein, a combination of models were used to analyze the data from the FirePlex® experiments. As discussed, a number of the models gave similar results and so a combination of models produced a higher degree of accuracy in determining the presence or absence of disease.

There is therefore provided an miRNA assay to accurately identify the presence or absence of mycobacterial disease, such as Johne's disease, in a mammalian subject (such as cows) using a biofluid such as a blood sample.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

### REFERENCES

Benedictus G., Dijkhuizen A.A., Stelwagen J.Economic losses due to paratuberculosis in dairy cattle. 1987. Veterinary Record 121: 142-146.
Bennett R, IJpelaar J. Updated estimates of the costs associated with thirty four endemic livestock diseases in Great Britain: A note. J Agric Econ 2005;56(1):135-144.
Coutinho LL, Matukumalli LK, Sonstegard TS, Van Tassell CP, Gasbarre LC, Capuco AV, Smith TP. Discovery and profiling of bovine microRNAs from immune-related and embryonic tissues. Physiol Genomics. 2007 Mar. 14;29(1):35-43.
Defra, ADAS Study to Model the Impact of Controlling Endemic Cattle Diseases and Conditions on National Cattle Productivity, Agricultural Performance and Greenhouse Gas Emissions 2015.
Dow CT, Sechi LA. Cows Get Crohn's Disease and They're Giving Us Diabetes. Microorganisms. 2019 Oct. 17;7(10):466.
Giannopoulou L, Zavridou M, Kasimir-Bauer S, Lianidou ES. Liquid biopsy in ovarian cancer: the potential of circulating miRNAs and exosomes. Transl Res. 2019 March;205:77-91.
Jia S, Zhai H, Zhao M. MicroRNAs regulate immune system via multiple targets. Discov Med. 2014 November; 18(100):237-47.
Lee RC, Ambros V. An extensive class of small RNAs in Caenorhabditis elegans. Science. 2001 Oct. 26;294(5543):862-4.
Lee RC, Feinbaum RL, Ambros V. The C. elegans heterochronic gene lin-4 encodes small RNAs with antisense complementarity to lin-14. Cell. 1993 Dec. 3;75(5):843-54.
Lima JF, Cerqueira L, Figueiredo C, Oliveira C, Azevedo NF. Anti-miRNA oligonucleotides: A comprehensive guide for design. RNA Biol. 2018 Mar. 4;15(3):338-352.
Matthews C, Cotter PD, O'Mahony J. MAP, Johne's disease and the microbiome; current knowledge and future considerations. Anim Microbiome. 2021 May 7;3(1):34.
McNees AL, Markesich D, Zayyani NR, Graham DY. Mycobacterium paratuberculosis as a cause of Crohn's disease. Expert Rev Gastroenterol Hepatol. 2015;9(12):1523-34.
Mehta A, Baltimore D. MicroRNAs as regulatory elements in immune system logic. Nat Rev Immunol. 2016 Apr. 28; 16(5):279-94.
Momen-Heravi F, Bala S. miRNA regulation of innate immunity. J Leukoc Biol. 2018 Apr. 14.
Ott S.L., Scott J. Wells S.J., Wagner, B.A. 1999. Herd-level economic losses associated with Johne's disease on US dairy operations. Preventive Veterinary Medicine 40: 179-192.
Rupaimoole R, Slack FJ. MicroRNA therapeutics: towards a new era for the management of cancer and other diseases. Nat Rev Drug Discov. 2017 March; 16(3):203-222.
Somoskovi A, Dormandy J, Parsons LM, Kaswa M, Goh KS, Rastogi N, Salfinger M. Sequencing of the pncA gene in members of the Mycobacterium tuberculosis complex has important diagnostic applications: Identification of a species-specific pncA mutation in “Mycobacterium canettii” and the reliable and rapid predictor of pyrazinamide resistance. J Clin Microbiol. 2007 February;45(2):595-9.
Tiwari and others 2003. Seropositivity for Bovine Leukaemia Virus, Mycobacterium avium subspecies paratuberculosis and Neospora caninum on the calving to conception interval in maritime Canadian dairy cattle. Proc Soc Veterinary Epidemiological Preventative Medicine p243
Valentin-Weigand P, Goethe R. Pathogenesis of Mycobacterium aviumsubspecies paratuberculosis infections in ruminants: still more questions than answers. Microbes Infect. 1999 November;1(13):1121-7.
Wightman B, Ha I, Ruvkun G. Posttranscriptional regulation of the heterochronic gene lin-14 by lin-4 mediates temporal pattern formation in C. elegans. Cell. 1993 Dec. 3;75(5):855-62.
Woodbine, K.A., Schukken, Y.H., Green, L.E., Ramirez-Villaescusa, A, Mason, S, Moore, S.J., Bilbao, C., Swann, N and Medley, G (2009) Seroprevalence and epidemiological characteristics of Mycobacterium avium subspecies paratuberculosis on 114 cattle farms in south west England. Preventative Veterinary Medicine 89 (1-2) 102-109.

What is claimed is:

1. A method for detecting the presence of mycobacterial infection and disease in a subject, comprising the steps of:
(a) determining a level of expression of each of a plurality of miRNA molecules within a sample from a subject; and
(b) comparing the level of expression of each miRNA molecule with at least one pre-determined reference level characteristic of a non-diseased subject for each one of the plurality of the miRNA molecules of step (a),
wherein a deviation of the level of expression of said miRNA molecules from step (a) in comparison with the at least one reference level allows for the diagnosis and/or prognosis of the disease.

2. The method according to claim 1, wherein the plurality of miRNA molecules is selected from a group consisting of bta-miR-19b, bta-miR-196b, bta-miR-146a, bta-miR-29b, bta-miR-29a, bta-miR-142-3p, bta-miR-155, bta-miR-582, bta-miR-21-5p, bta-miR-6517, bta-miR-7857-5p, bta-miR-24, bta-miR-378c, bta-miR-1271, bta-miR-100, bta-miR-301a, bta-miR-32, bta-miR-1247-5p, bta-miR-184, bta-miR-202, bta-miR-137, bta-miR-105a, bta-miR-433, bta-miR-133b, bta-miR-93, bta-miR-20a-5p, bta-miR-16a-5p, bta-miR-17-5p and bta-miR-92a; with oan-miR-7417-5p, cel-miR-70-3p and ath-miR-167d as off-species controls.

3. The method according to claim 1, wherein the subject is a mammal.

4. The method according to claim 4, wherein the subject is a cow, sheep, goat, deer, llama, alpaca or vicuna.

5. The method according to claim 1, wherein the method further comprises the use of at least one normaliser and/or control miRNA molecule.

6. The method according to claim 8, wherein the control miRNA molecule is an off-species control miRNA molecule.

7. The method according to claim 8, wherein the at least one normalizer is selected from a group consisting of bta-miR-93, bta-miR-16a-5p, bta-miR-17-5p and/or bta-miR-92a. 8 The method according to any one of claim 9, wherein the at least one off-species control is selected from a group consisting of oan-miR-7417-5p, cel-miR-70-3p and/or ath-miR-167d.

9. The method according to claim 1, wherein the disease is selected from a group consisting of mycobacterial disease, including but not restricted to Johne's disease, tuberculosis and Crohn's disease, or any other disease caused by Mycobacterium avium subspecies paratuberculosis (MAP) or any other species within the Mycobacterium genus.

10. The method according to claim 1, wherein the sample is a biofluid selected from the group consisting of blood, urine, milk, tissue fluid, saliva, cerebrospinal fluid (CSF), faeces or another biofluid.

11. The method according to claim 1, wherein the miRNAs are cell free miRNAs.

12. A kit for use in performing the method of claim 1 comprising means for determining the level of expression of each one of the following miRNA molecules: bta-miR-19b, bta-miR-196b, bta-miR-146a, bta-miR-29b, bta-miR-29a, bta-miR-142-3p, bta-miR-155, bta-miR-582, bta-miR-21-5p, bta-miR-6517, bta-miR-7857-5p, bta-miR-24, bta-miR-378c, bta-miR-1271, bta-miR-100, bta-miR-301a, bta-miR-32, bta-miR-1247-5p, bta-miR-184, bta-miR-202, bta-miR-137, bta-miR-105a, bta-miR-433, bta-miR-133b, bta-miR-93, bta-miR-20a-5p, bta-miR-16a-5p, bta-miR-17-5p, bta-miR-92a, oan-miR-7417-5p, cel-miR-70-3p and ath-miR-167d.

13. A method of selecting a panel for use in disease diagnosis comprising the steps of:
(a) selecting a group of miRNA molecules the differential expression of which may be associated with a disease condition;
(b) predicting the disease condition based on a deviation of the level of expression of said miRNA molecules from step (a) and (b); and
(c) reducing the number of miRNAs in the panel to a minimum number to provide a panel of miRNAs that still produces a result.

14. A method of selecting a panel for use in differential diagnosis between a symptom-free M. paratuberculosis infected subject and a symptomatic M. paratuberculosis infected subject comprising the steps of:
(a) selecting a group of miRNA molecules the differential expression of which may be associated with a disease's symptomatic state;
(b) predicting the disease's symptomatic state; and
(c) reducing the number of miRNAs in the panel to a minimum number to provide a panel of miRNAs that still produces a result.
